(19) United States
(12) Patent Application Publication
(10) Pub. No.: US 2010/0152783 A1
(43) Pub. Date: Jun. 17, 2010

(54) Universal Surgical Plate with 30 Degree Compression Angle

(75) Inventors: Frank Borostyankoi, Huntington Beach, CA (US); Jeffrey Norton, Manassas, VA (US)

Correspondence Address: Law Offices of Daniel L. Dawes;Dawes Patent Law Group, 5200 Warner Blvd, Ste. 106, Huntington Beach, CA, 92649, US

(73) Assignee: VETERINARY IMPLANTS DIRECT, LLC, Huntington Beach, CA (US)

(21) Appl. No.: 12/635,930

(22) Filed: Dec. 11, 2009

Related U.S. Application Data
(60) Provisional application No. 61/121,863, filed on Dec. 11, 2008.

(51) Int. Cl. A61B 17/88; A61B 17/80

(52) U.S. Cl. 606/281; 606/70

(57) ABSTRACT

A universal surgical plate comprises a symmetrical design which enables it to be used for both right and left sided TPLO procedures as well as other orthopedic procedures on other bilaterally symmetric bones. The plate provides a 30 degree angle compression force with respect to the longitudinal axis of the plate in order to ensure maximum compression across an osteotomy or fracture. The plate is affixed across the osteotomy or fracture with orthopedic screws disposed through a plurality of elongated and circular drive apertures. The drive apertures are defined within the plate at sufficient distances to preserve the integrity of the underlying bone. The body of the plate also comprises a plurality of notches for improving periosteal circulation. The head of the plate includes a pair of recessed corners so that the plate may be implanted close to an assisting jig pin on smaller breeds of canines.

### RELATED APPLICATIONS

The present application is related to U.S. Provisional Patent Application, Ser. No. 61/121,863, filed on Dec. 11, 2008, which is incorporated herein by reference and to which priority is claimed pursuant to 35 USC 119.

### BACKGROUND OF THE INVENTION

1. Field of the Invention

The invention relates to the field of orthopedic surgical implants for animals, specifically orthopedic surgical implants for use during tibial plateau leveling osteotomy (TPLO).

2. Description of the Prior Art

When the cranial cruciate ligament of an animal such as a canine ruptures, the stifle joint of the animal becomes unstable and causes a decrease in mobility and an increase in arthritis. To correct this condition, a procedure well known in the art called tibial plateau leveling osteotomy (TPLO) is performed which keeps the stifle joint stable as well as prevents the femur from sliding down on the slope of the tibial plateau during weight bearing.

Several devices well known in the art have long been used to help the veterinary professional perform a successful TPLO procedure. Typically, these devices consist of a surgical plate with a plurality of screw holes defined therein that help couple the plate to the proximal tibia and the metaphysis of the tibia. Once implanted, the plate holds the previously cut bones in a new position while the animal heals.

However while many of these plates are not without their various degrees of success, several limitations or drawbacks have been found over time which severely limit their use. For example, the TPLO plates are leg specific, such as that shown by Slocum, U.S. Pat. No. 5,304,180, which is incorporated herein by reference as if set out in its entirety. That is, one type of plate is used for the rear right leg of the canine and a separate and differently orientated plate is used for the rear left leg. This effectively doubles the required inventory of the veterinary surgeon. It is also taught in Slocum that it is absolutely necessary to achieve the desired stability of the bone segments during the TPLO procedure with the use of specifically angled compressive screw holes. Although there have been attempts with some success to perform the TPLO procedure with no specific implants, there is as yet to date no way to provide the desired compression angels for a balanced osteotomy stabilization while the same plate is used on either left or right tibias. Without the proper forward compression, the proximal cut bone segment is subject to shift caudally due to the loading forces and muscle action forces, creating a phenomenon called a “rock back”. During proximal fragment shift “rock back,” the cranial end of the proximal tibia and the tibial crest becomes unsupported from losing contact of the now caudally displaced proximal fragment, resulting in a possible tibial crest fracture. Complications such as proximal fragment destabilization or tibial crest fracture could lead to a catastrophic failure of the procedure. Therefore balanced osteotomy stabilization with downward and forward compression is essential for a successful TPLO procedure. Additionally, there are specific canine breeds they have a relative large body weight carried on a small bone structure. These canines have a very high incident of cranial cruciate ligament rupture. The large body mass requires strong plates and larger screws to be installed while the available bone is limited in size to accommodate the stronger implants. Finally, most of the TPLO plates found in the prior art can only be used for TPLO procedures or in some proximal tibia fractures. There as of now no universal surgical bone plate that may also accommodate other fracture repairs such as an ilial fracture where a space conserving shape and a series of preset compression angles are desired.

What is needed is a universal surgical plate that may be implanted on either the right or left legs of a canine during a TPLO procedure, or for any number of other surgical bone procedures, for any type or breed of canine.

### BRIEF SUMMARY OF THE INVENTION

The present invention is a universal surgical bone plate used in veterinary orthopedics, which means that where the anatomical bilateral symmetry of the skeleton presents a bone in bone a left and right configuration, or a bone which has left or right portions, the universal surgical bone plate can be used without substantial or without any modification other than minor manual bending or twisting, if any, to be optimally implantable on either the left or right configuration or portion of the surgically mediated bone.

Tibial Plateau Leveling Osteotomy (TPLO) is a well known orthopedic technique for treatment of cranial cruciate ligament rupture in canines, as shown and described, for example, in Slocum, U.S. Pat. No. 5,304,180. During a TPLO procedure, the slope of the tibial plateau is reduced causing tibial forces on the stifle joint to shift from the cranial toward the caudal end with a greater reliance on the caudal cruciate ligament for stability. The illustrated embodiment of the invention is directed to a new bone fixation plate to be used in canine tibial plateau leveling osteotomy surgical procedures and difficult fracture repairs where the unique shape of the plate is desired. During a TPLO procedure, the improved bone fixation plate promotes fastening of the bone sections resulting from an optimal centered osteotomy. With a centered osteotomy, the long axis of the tibia remains in the same position, and the surgeon may accurately achieve the desired post operative tibial plateau angle to counter act the cranial tibial thrust. Many anatomical changes are avoided with the centered osteotomy.

An aspect of the illustrated embodiment of the invention is a bone fixation plate for the tibial plateau leveling osteotomy that works with the principals of dynamic compressive fixation of bone segments. The symmetrical design allows the plate to be used on both left and right rear legs.

The design of the current surgical plate of the illustrated embodiment allows it to also be placed against assisting jig pins for improved positioning and a better fitting on a smaller proximal fragment that may be present with smaller breeds of canines. The contour of the implant is tailored to suit the profile of the underlying bone. The placement of the staggered apertures defined within the plate improves compression of the osteotomy. The plate has six holes or apertures defined in it to allow the use of orthopedic screws to secure the plate over the osteotomy and to create the compression forces. Forward compression is supported at a 30 degree compression angle through a selected one of two symmetrically defined drive apertures in the plate. The position and number of drive apertures optimizes the fastening of the plate to the underlying bone. The drive and anchor apertures in the plate are recessed to accommodate conventional cortical and cancellous orthopedic screws.

The plate has sufficient strength to carry the loads applied to the affected bone during movement after implantation, yet the plate is produced from a malleable material which is able to accept additional manual contouring by the surgeon.

Furthermore, the plate has four recessed notches on the underside of the plate to provide minimal circulatory compromise to the underlying bone.

Because of its symmetrical design, the plate may be used in other areas of veterinary orthopedic surgery. For example, in the case of a canine hip fracture, the plate may be implanted across the fracture, thus coupling the bone together until healing has been completed.

The current invention is a universal surgical plate having a longitudinal axis for implantation in a canine during an orthopedic procedure wherein two segments of a bone having bilateral anatomical symmetry are fixed as a whole and wherein the two segments of the bone are produced by a curvilinear cut, the plate comprising a head portion symmetrically shaped with respect to the longitudinal axis comprising at least three elongated drive apertures symmetrically defined in the head portion with respect to the longitudinal axis, each of the at least three elongated drive apertures having a different drive axes oriented generally normal to the curvilinear cut in the bone, two of the corresponding drive axes being symmetrically inclined with respect to the longitudinal axis, and a body portion extending along the longitudinal axis and operable with the head portion and including an anchor aperture, where selected one of the at least three elongated drive apertures include a fastener received within and through the corresponding drive aperture and disposed into one of the two bone segments, and being operable, depending on the location of the fastener within the aperture, to selectively and independently to urge compression and distraction of the bone segments along the corresponding drive axis of the selected apertures, whereby the universal surgical plate may be implanted on the segments of the bone regardless of which bilateral anatomical symmetry characterizes the bone.

In one particular embodiment, the surgical plate further comprises a neck portion connecting the head portion and the body portion.

In another embodiment, the body portion of the surgical plate further comprises an elongated drive aperture defined therein, and at least two circular shaped anchor apertures defined therein. Additionally, the body portion may further comprise where at least one of the three elongated drive apertures defined in the head portion and the elongated drive aperture defined in the body portion are arranged and configured to apply equal and opposing compression forces along the longitudinal axis when the corresponding fasteners are received therein. Alternatively, the at least three elongated drive apertures defined in the head portion and the elongated drive aperture defined in the body portion are each arranged and configured as means for providing compression on the curvilinear cut when coupled to the underlying bone when the corresponding fastener is received therein.

In another embodiment, the surgical plate includes a front and reverse side, and where the body portion of the plate further comprises a plurality of notches defined on the reverse side of the body portion that reduce surface contact between the plate and underlying bone.

In yet another embodiment, the head portion of the surgical plate includes an end and further comprises a pair of recessed corners provided on the end thereof.

In an alternative embodiment, the surgical plate comprises where at least two of the three elongated drive apertures defined in the head portion are arranged and configured to apply a compression force to the curvilinear cut in a direction 30 degrees with respect to either side of the longitudinal axis when the corresponding fastener is received therein.

In still another embodiment, each drive aperture of the surgical plate has a distal end and where the at least three elongated drive apertures defined in the head portion are defined within the head portion so that the distal end of each drive aperture is defined at a maximum distance from every other drive aperture distal end in the head portion.

The current invention also provides for a method of implanting a universal surgical plate during a tibial plateau leveling osteotomy (TPLO) procedure in either the left or right rear leg of a canine. The method comprises making a curvilinear cut in a left or right tibia to define at least two bone segments, inserting a jig pin into one of the at least two bone segments of the tibia above the curvilinear cut for coupling to a surgical fixture to stabilize the corresponding bone segment, and coupling the universal surgical plate to the stabilized segment of the left or right tibia close to the jig pin and the other of the at least two bone segments with a plurality of orthopedic screws and straddling the curvilinear cut. Finally, the method comprises applying a selectively directed compressive force 30 degrees with respect to a longitudinal axis of the surgical plate across the curvilinear cut by fastening an orthopedic screw into the stabilized segment into a selected one of two drive apertures which are symmetrically defined in the plate with respect to the longitudinal axis of the plate, which selected one of the symmetrically defined apertures is selected depending on whether the plate is implanted onto the left or right tibia.

In one particular embodiment, the method of coupling the surgical plate to the tibia close to the jig pin comprises coupling a recessed corner of the surgical plate substantially close to the jig pin.

In another embodiment, the method of coupling the surgical plate to the tibia comprises inserting at least two compressive drive orthopedic screws above the curvilinear cut, inserting at least one anchor orthopedic screw above the curvilinear cut, inserting at least one compressive drive orthopedic screw below the curvilinear cut, and inserting at least two anchor orthopedic screws below the curvilinear cut.

In yet another embodiment, the method above comprises applying a directed compressive force 30 degrees to the left of the longitudinal axis of the surgical plate when the surgical plate is implanted on the right rear leg of the canine as seen from a front side of the plate with a head of the plate at the top.

The method in an alternative embodiment comprises applying a directed compressive force 30 degrees with respect to a longitudinal axis of the surgical plate which comprises applying a directed compressive force 30 degrees to the right of the longitudinal axis of the surgical plate when the surgical plate is implanted on the left rear leg of the canine as seen from a front side of the plate with a head of the plate at the top.

In another embodiment the method further comprises applying a directed compressive force along the longitudinal axis and an equal and opposite directed compressive force along the longitudinal axis.

The current invention also provides for a second method of implanting a universal surgical plate having a longitudinal axis for treating a bone fracture in a canine wherein the bone has bilateral anatomical symmetry comprising coupling a head portion of the universal surgical plate to the bone with a plurality of orthopedic screws on a side the fracture which is characterized by a left or right bilateral anatomical symmetry by selectively fastening to the bone through a selected one of at least two symmetrically defined angularly oriented drive apertures in the head portion, which drive apertures are symmetrically defined with respect to the longitudinal axis, disposing a neck portion of the universal surgical plate across the fracture, and coupling a body portion of the universal surgical plate to the bone on an opposing side of the fracture with a plurality of orthopedic screws.

In another embodiment, the method step of where coupling the head portion of the surgical plate with a plurality of orthopedic screws comprises inserting at least one compressive force orthopedic drive screw into the surgical plate so that a directed compressive force 30 degrees from the longitudinal axis of the body portion is applied to the bone.

In yet another embodiment, the method step of coupling the head portion of the surgical plate with a plurality of orthopedic screws comprises inserting at least one compressive force orthopedic drive screw into the surgical plate so that a directed compressive force along the longitudinal axis of the body is applied.

In still another embodiment, the method step of coupling the body portion of the surgical plate with a plurality of orthopedic screws comprises inserting at least one compressive force orthopedic drive screw into the surgical plate so that a directed compressive force along the longitudinal axis of the body is applied.

Finally, in another embodiment, the method further comprises reducing the surface contact between the surgical plate and underlying by means of a plurality of notches defined into the reverse side of the body portion.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a front plan view of the illustrated embodiment of the surgical plate with the apertures shown in light outline, the potential relative initial and final positions of the corresponding screw or fastener received in the aperture shown in bold outline, the position of the anchor fasteners shown in double dotted outline.

FIG. 2 is rear plan view of the surgical plate in FIG. 1.

FIG. 3 is a medial view of a canine's proximal tibial region with the surgical plate of the illustrated embodiment implanted to the right proximal tibia.

FIG. 4 is a medial view of a canine's proximal tibial region with the surgical plate of the illustrated embodiment implanted to the left proximal tibia.

FIG. 5 is a perspective view of a right canine pelvic bone with the surgical plate of the illustrated embodiment implanted ilial shaft fracture.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

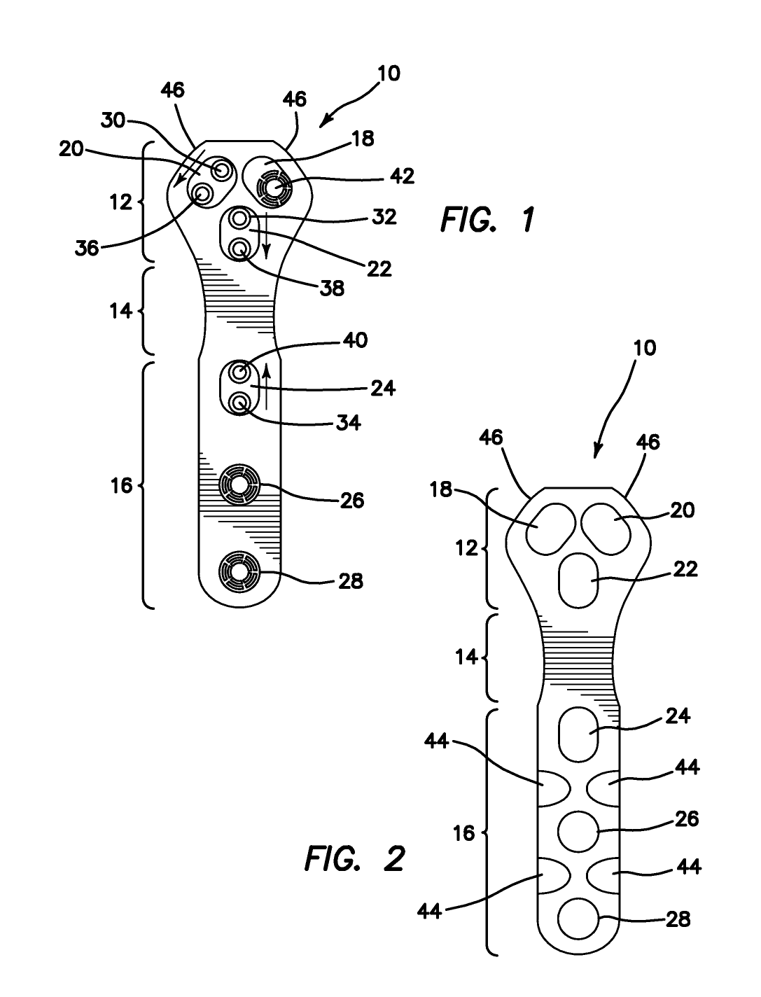

Referring now to the illustrated embodiment of the invention in more detail, in FIGS. 1 and 2 there is shown a universal plate, generally denoted by reference numeral 10. The plate 10 is comprised of three main parts or portions including a head 12, a neck 14, and a body 16. Defined within the plate 10 are six drive apertures, a right proximal aperture 18, a left proximal aperture 20, a center proximal aperture 22, a first body aperture 24, a second body aperture 26, and a third body aperture 28.

The right proximal aperture 18, left proximal aperture 20, center proximal aperture 22, and first body aperture 24 are substantially elongated or oval along an elongate axis, and the second body aperture 26 and third body aperture 28 are substantially circular in shape. In the head 12, both the right and left proximal apertures 18, 20 are defined within the plate 10 so that the elongate axis of each aperture 18, 20 is orientated 30 degrees from the longitudinal axis of the plate 10. The right proximal aperture 18 is orientated 30 degrees to the right from the longitudinal axis of the plate 10, while the left proximal aperture 20 is orientated 30 degrees to the left of the longitudinal axis of the plate 10 as seen in the illustration in FIG. 1, so that the elongate axes of apertures 18 and 20 are oriented 60 degrees apart from each other.

Preferably, the total length of the plate 10 of the illustrated embodiment is 60 mm. The width of the head 12 of the plate 10 is 18.5 mm, the narrowest portion of the neck 14 of the plate is 8.5 mm and the body 16 of the plate 10 is 10.4 mm wide. It is to be expressly understood that similar proportions may be used as long as the right and left proximal apertures 18, 20 on the head 12 are each angled at approximately 30 degrees with respect to the longitudinal axis of the plate 10 and are symmetrically oriented with respect to each other along the longitudinal axis. It is to be further understood that the magnitude of angular orientation with respect to the longitudinal axis of plate 10 may be varied as required by the particular application to which the plate 10 is applied.

The plate 10 is preferably comprised of 316L or 316LVM medical grade steel or any other sufficiently strong but malleable metal which is biocompatible that is known in the art, such as titanium. The material which comprise the plate 10 must be rust free, biocompatible, and malleable for further manual shaping, bending and twisting prior to installation but strong enough in its implanted configuration to counteract the relevant forces from the movements of the patient. The plate 10 should be constructed from materials pre-manufactured for orthopedic use and further machined in accordance standards currently known in the art. Surface treatments of the plate 10 may include, but are not limited to polish, matte or other coatings by now known or later devised related to that of implant manufacture. The plate 10 is also radio-dense so that its positioning and integrity may be evaluated in association with common radiographic techniques such as x-ray.

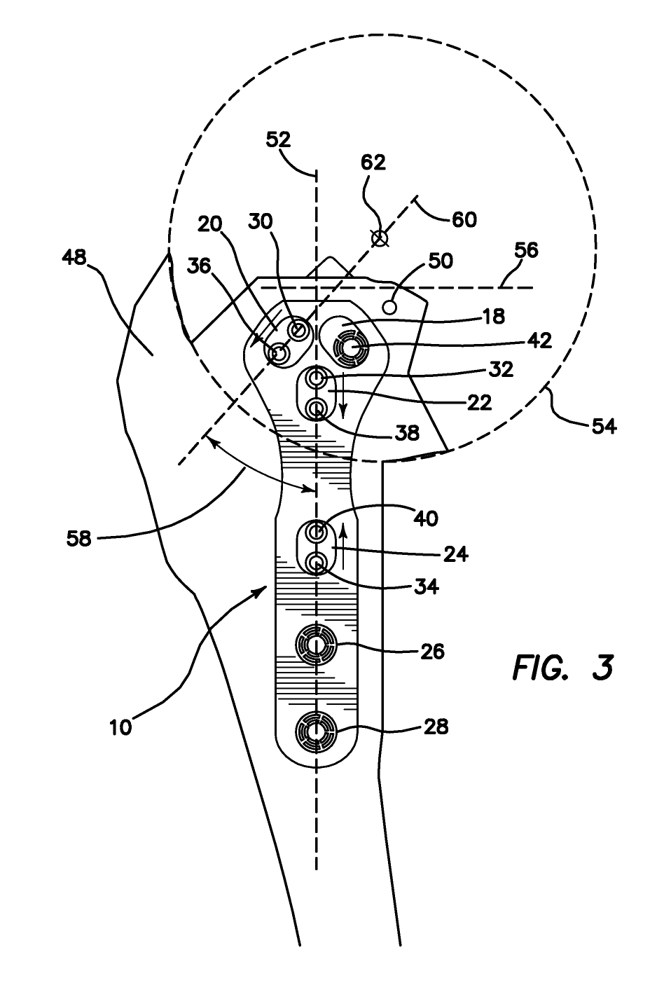

When the plate 10 is implanted onto the right rear leg of the canine as seen in FIG. 3, primary drill apertures 30, 32, 34 are positioned in the bone prior to screw installation at the places where compression coupling is to take place, namely the areas where the left proximal aperture 20, center proximal aperture 22, and the first body aperture 24 are to be placed when the plate 10 is implanted. The arrows indicated in FIG. 1 are the directions of the fragmentary bone shift under plate 10 towards the osteotomy site in accordance with the well known principles of dynamic compression during the screw tightening process. In the right proximal aperture 18, an orthopedic screw is placed in non-compressive fashion as is known in the art in the selected position 42 within the aperture 18 as well as in the second and third body apertures 26, 28. The center proximal and first body aperture provide a vertical downward and upward compression force respectively, while the left proximal aperture 20 provides a downward and forward force 30 degrees from the longitudinal axis of the plate 10 that is critical to treating a CCL rupture in canines.

At this point it is to be expressly understood that plate 10 is a universal plate and due to the symmetry of the right and left proximal apertures 18, 20, it may be implanted during other orthopedic procedures where directional compression of bone segments is required. For example, the plate 10 may be implanted on the right rear leg of a canine using the same orthopedic screw configuration described above except for that a compressive screw is inserted in the right proximal aperture 18, and a non-compressive screw is placed in the left proximal aperture 20. In situations where directional forces 30 degrees from both sides of the longitudinal axis of the plate 10 are needed, compressive screws may be placed in both the right and left proximal apertures 18, 20. Alternatively, non-compressive screws may be placed in the right and left apertures 18, 20 when no directional compression is required. Furthermore it is to be expressly understood that all compressive fashion screw apertures 20, 22, 24 can be utilized in a none compressive fashion with a corresponding plurality of secondary drill apertures positions 36, 38, 40 where no directional compression is desired, one secondary drill aperture being disposed within each of the elongated drive apertures 20, 22, 24 which are to provide a compressive force.

Turning now to FIG. 2, the back view of the universal plate 10 is shown. Defined into the back surface of the plate 10, are a plurality of recessed notches 44 which enable the plate 10 to have minimal contact with the underlying bone surface and improve periosteal circulation. While four recessed notches 44 are shown in FIG. 2, it is to be expressly understood that fewer or more notches 44 or other types of surface relief cavities may be used without departing from the original spirit and scope of the invention.

Also defined within the head portion 12 of the plate 10 are a pair of sloped or recessed corners 46. Because of the symmetric orientation of the right and left proximal apertures 18, 20, the recessed corners 46 are defined substantially close to the edge of the apertures 18, 20 thus cutting down on the overall size of the head 12. The recessed corners 46 not only reduce the overall size and weight of the plate 10, but allows the plate 10 to be more effectively placed within the implant site as will be detailed below.

Referring now to FIG. 3, the universal plate 10 is shown implanted on a right proximal tibia 48 during a TPLO procedure. During most routine TPLO procedures, an assisting jig pin 50 is implanted into the tibia 48 to temporarily stabilize the proximal tibia 48 segment during the surgical procedure.

As described above, the plate 10 is configured with multiple elongated apertures 20, 22, 24 defined therein for receiving orthopedic screws that are compressive, forcing the universal plate 10 when coupled to the bone 48 to produce a downward and forward 30 degree angle of compression relative to a longitudinal axis 52 of the plate 10 represented by a single dotted line in FIG. 3 to ensure maximum compression along a semi-circular osteotomy 54 cut in accordance with the practice of the proximal tibial osteotomy. Apertures 18, 26, 28 are also defined therein for receiving orthopedic screws in a static locking position.

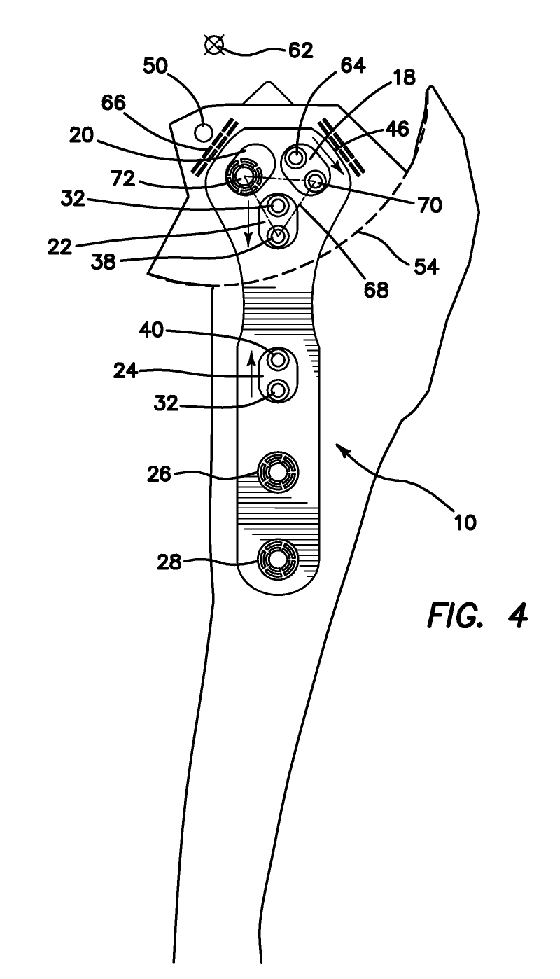

In more detail, the universal plate 10 when used in a TPLO procedure is affixed to the right proximal tibia 48 with six orthopedic screws to maintain a desired new tibial plateau angle 56 for the period of bone healing at the osteotomy site. Prior to affixing the plate 10, several routine steps are taken that are well known that execute a tibial plateau angle decrease usually, but not exclusively to 0-6 degrees. The plate 10 should be contoured to the shape of the medial aspect of the osteotomy site with the use of any orthopedic plate benders and twisters known in the art. The plate 10 is then affixed to the osteotomy site with standard orthopedic screws in the fashion shown in FIGS. 3 and 4. The elongated shape of the dynamic compression drive apertures 18, 20, 22, 24 can be filled with screws providing approximately 1 mm shift of the underlying bone in the direction of the osteotomy as indicated by the arrows shown in FIGS. 3 and 4. Such a shift may be necessary to facilitate proper compression at the osteotomy site to achieve superior stability of the participating bone fragments and achieve primary bone healing as described in the orthopedic literature.

In order to achieve such a compression, screw installation within the plate 10 has to be initiated away from the osteotomy site, that is at primary drill apertures 30, 32, 34. The left proximal aperture 20, center proximal aperture 22, and first body aperture 24 will then shift the underlying bone 48 towards the osteotomy site during the installation of the orthopedic screws. The dynamic compression of the bone fragments occurs along the longitudinal axis 52 of the plate 10 by the center proximal aperture 22 and first body aperture 24 and in a 30 degree angle 58 relative to the longitudinal axis 52 of the plate 10 from the left proximal aperture 20. The compression force applied by the left proximal aperture 20 is perpendicular to the semi-circular osteotomy 54 and also radially aligned with the center 62 of the circular osteotomy 54 with its center of rotation at the mobilized proximal bone segment as shown in FIG. 3.

The elongated dynamic compression drive apertures 18, 20, 22, 24 can also be filled with regular orthopedic screws in a non-shifting fashion when compression is not needed. In this embodiment, orthopedic screws are placed against the plate 10 in each portion of the elongated drive apertures 20, 22, 24 that is closest to the osteotomy site 54 at secondary drill apertures 36, 42, 38, 40. In direct contrast, second and third body apertures 26, 28 are circular apertures that can only be used with orthopedic screws in a locking fashion.

Due to the different functional capabilities of the elongated drive apertures 18, 20, 22, 24, i.e. compressive and non-compressive, the order in which the orthopedic screws are placed also greatly influences the results of the procedure. For the configuration depicted in FIG. 3, preferably the primary drill aperture 32 within the center proximal aperture 22 should receive the first orthopedic screw, followed then by primary drill aperture 34 within the first body aperture 24, the third body aperture 28, the second body aperture 26, the primary drill aperture 30 within the left proximal aperture 20, and then finally the selected position 42 within the right proximal aperture 18. At this point it is important to point out that it is well known in orthopedics that when using dynamic compression apertures, intra-fragmentary bone shift during screw installation only starts at the time of the screw head reaching contact with the orthopedic bone plate while advancing the bone screw. The operator has the advantage of staging the advancement of the screws during installation in a staged fashion to achieve the desired maximum shift or compression.

It is in this fashion that any number of compressive and non-compressive configurations may be applied according the application in which the plate 10 is being applied. It is to be expressly understood that other configurations other than what is described here may be used without departing from the original spirit and scope of the invention.

Turning now to FIG. 4, the universal plate 10 is shown implanted on the left proximal tibia during a TPLO procedure. Like implantation on the right proximal tibia 48 shown in FIG. 3, an assisting jig pin 50 is placed which is routinely used in TPLO procedures to temporarily stabilize the proximal bone segment during the surgical procedure. FIG. 4 also shows how the plate 10 is configured with the plurality of elongated drive apertures 18, 20, 22, 24 defined therein for applying compressive forces in the directions indicated as well as circular apertures 26, 28 defined therein for coupling the plate 10 to the bone in a fixed, non-compressive position.

As described above the head 12 portion of the plate 10 comprises a pair of recessed corners 46. The recessed corners not only more accurately replicate the shape of the underlying bone, they also allow for the plate 10 to be implanted in close proximity to the jig pin 50 with the interlaying space represented by dotted lines denoted with reference numeral 66. The ability to implant the plate 10 close to the jig pin 50 is particularly advantageous in smaller canines where space is limited.

The primary and secondary drill apertures in the embodiment shown in FIG. 4 are substantially the same as those in the embodiment of FIG. 3 except that the right proximal aperture 18 now comprises a primary drill aperture 64 and a secondary drill aperture 70 and the left proximal aperture 20 only comprises a primary drill aperture 72. This configuration is established because in the current embodiment, it is the right proximal aperture 18 that provides a compressive force 30 degrees from the longitudinal axis of the plate 10, rather than the left proximal aperture 20 as disclosed in the previous embodiment.

Another aspect of the plate 10 is that within the head 12 of the plate 10, the primary drill aperture 72 of the left proximal aperture 20, the secondary drill aperture 70 of the right proximal aperture 18, and the secondary drill aperture 38 of the center proximal aperture 22 are at a maximum distance 68 from each other. Having a plurality of orthopedic screws at the maximum distance 68 from each other as is allowed by the current configuration of elongated drive apertures 18, 20, 22 helps to preserve the integrity of the underlying bone.

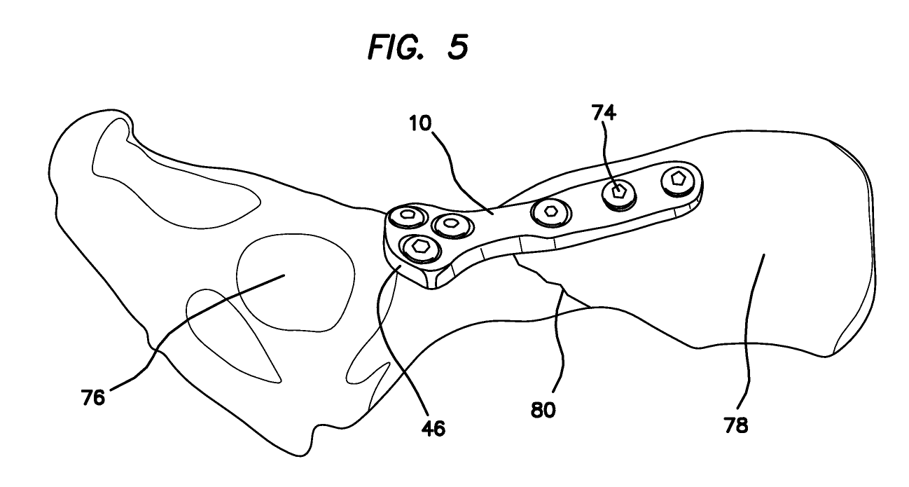

In another embodiment, the plate 10 may be used in a variety of orthopedic procedures other than TPLO, for example for hip fractures as depicted in FIG. 5. Here, the plate 10 is implanted to a hip bone 78 over a fracture 80 with a plurality of orthopedic screws 74 as is described above. The space saving configuration of the head 12 and the recessed corners 46 of the plate 10 allow the plate 10 to be closely placed to the hip socket 76, thus enabling use of the plate 10 on smaller breeds of canines where bone surface area is at a premium.

In summary, the illustrated embodiment of the invention is a universal surgical plate having a longitudinal axis for implantation in a canine during an orthopedic procedure wherein two segments of a bone having bilateral anatomical symmetry are fixed as a aperture and wherein the two segments of the bone are produced by a curvilinear cut, the plate comprising: a head portion symmetrically shaped with respect to the longitudinal axis comprising at least three elongated drive apertures symmetrically defined in the head portion with respect to the longitudinal axis, each of the at least three elongated drive apertures having a different drive axes oriented generally normal to the curvilinear cut in the bone, two of the corresponding drive axes being symmetrically inclined with respect to the longitudinal axis; and a body portion extending along the longitudinal axis and operable with the head portion and including an anchor aperture, where selected ones of the at least three elongated drive apertures include a fastener received within and through the corresponding drive aperture and disposed into one of the two bone segments, and being operable, depending on the location of the fastener within the aperture, to selectively and independently to urge compression and distraction of the bone segments along the corresponding drive axis of the selected apertures, whereby the universal surgical plate may be implanted on the segments of the bone regardless of which bilateral anatomical symmetry characterizes the bone.

The surgical plate further comprises a neck portion connecting the head portion and the body portion.

The body portion of the plate further comprises: an elongated drive aperture defined therein; and at least two circular shaped anchor apertures defined therein.

The plate includes a front and reverse side, and where the body portion of the plate further comprises a plurality of notches defined on the reverse side of the body portion that reduce surface contact between the plate and underlying bone.

The head portion includes an end and further comprises a pair of recessed corners provided on the end thereof.

In the illustrated embodiment at least two of the three elongated drive apertures defined in the head portion are arranged and configured to apply a compression force to the curvilinear cut in a direction 30 degrees with respect to either side of the longitudinal axis when the corresponding fastener is received therein.

The three elongated drive apertures defined in the head portion and the elongated drive aperture defined in the body portion are arranged and configured to apply equal and opposing compression forces along the longitudinal axis when the corresponding fasteners are received therein.

Each drive aperture has a distal end and the three elongated drive apertures defined in the head portion are defined within the head portion so that the distal end of each drive aperture is defined at a maximum distance from every other drive aperture distal end in the head portion.

The three elongated drive apertures defined in the head portion and the elongated drive aperture defined in the body portion are each arranged and configured as means for providing compression on the curvilinear cut when coupled to the underlying bone when the corresponding fastener is received therein.

The illustrated embodiment of the invention also includes within its scope a method of implanting a universal surgical plate during a tibial plateau leveling osteotomy (TPLO) procedure in either the left or right rear leg of a canine comprising the steps of: making a curvilinear cut in a left or right tibia to define at least two bone segments; inserting a jig pin into one of the at least two bone segments of the tibia above the curvilinear cut for coupling to a surgical fixture to stabilize the corresponding bone segment; coupling the universal surgical plate to the stabilized segment of the left or right tibia close to the jig pin and the other of the at least two bone segments with a plurality of orthopedic screws and straddling the curvilinear cut; and applying a selectively directed compressive force 30 degrees with respect to a longitudinal axis of the surgical plate across the curvilinear cut by fastening an orthopedic screw into the stabilized segment into a selected one of two drive apertures which are symmetrically defined in the plate with respect to the longitudinal axis of the plate, which selected one of the symmetrically defined apertures is selected depending on whether the plate is implanted onto the left or right tibia.

The step of coupling the surgical plate to the tibia close to the jig pin comprises coupling a recessed corner of the surgical plate substantially close to the jig pin.

The step of coupling the surgical plate to the tibia comprises the steps of: inserting at least two compressive drive orthopedic screws above the curvilinear cut; inserting at least one anchor orthopedic screw above the curvilinear cut; inserting at least one compressive drive orthopedic screw below the curvilinear cut; and inserting at least two anchor orthopedic screws below the curvilinear cut.

The step of applying a directed compressive force 30 degrees with respect to a longitudinal axis of the surgical plate comprises applying a directed compressive force 30 degrees to the left of the longitudinal axis of the surgical plate when the surgical plate is implanted on the right rear leg of the canine as seen from a front side of the plate with a head of the plate at the top.

The step of applying a directed compressive force 30 degrees with respect to a longitudinal axis of the surgical plate comprises applying a directed compressive force 30 degrees to the right of the longitudinal axis of the surgical plate when the surgical plate is implanted on the left rear leg of the canine as seen from a front side of the plate with a head of the plate at the top.

The method further comprises the step of applying a directed compressive force along the longitudinal axis and an equal and opposite directed compressive force along the longitudinal axis.

The illustrated embodiment of the invention still further includes a method of implanting a universal surgical plate having a longitudinal axis for treating a bone fracture in a canine wherein the bone has bilateral anatomical symmetry comprising the steps of: coupling a head portion of the universal surgical plate to the bone with a plurality of orthopedic screws on a side the fracture which is characterized by a left or right bilateral anatomical symmetry by selectively fastening to the bone through a selected one of at least two symmetrically defined angularly oriented drive apertures in the head portion, which drive apertures are symmetrically defined with respect to the longitudinal axis; disposing a neck portion of the universal surgical plate across the fracture; and coupling a body portion of the universal surgical plate to the bone on an opposing side of the fracture with a plurality of orthopedic screws.

The step of coupling the head portion of the surgical plate with a plurality of orthopedic screws comprises inserting at least one compressive force orthopedic drive screw into the surgical plate so that a directed compressive force 30 degrees from the longitudinal axis of the body portion is applied to the bone.

The step of coupling the head portion of the surgical plate with a plurality of orthopedic screws comprises inserting at least one compressive force orthopedic drive screw into the surgical plate so that a directed compressive force along the longitudinal axis of the body is applied.

The step of coupling the body portion of the surgical plate with a plurality of orthopedic screws comprises inserting at least one compressive force orthopedic drive screw into the surgical plate so that a directed compressive force along the longitudinal axis of the body is applied.

The method further comprises the step of reducing the surface contact between the surgical plate and underlying bone by means of a plurality of notches defined into the reverse side of the body portion.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. A universal surgical plate having a longitudinal axis for implantation in a canine during an orthopedic procedure wherein two segments of a bone having bilateral anatomical symmetry are fixed as a whole and wherein the two segments of the bone are produced by a curvilinear cut, the plate comprising:
a head portion symmetrically shaped with respect to the longitudinal axis comprising at least three elongated drive apertures symmetrically defined in the head portion with respect to the longitudinal axis, each of the at least three elongated drive apertures having a different drive axes oriented generally normal to the curvilinear cut in the bone, two of the corresponding drive axes being symmetrically inclined with respect to the longitudinal axis; and
a body portion extending along the longitudinal axis and operable with the head portion and including an anchor aperture,
where selected ones of the at least three elongated drive apertures include a fastener received within and through the corresponding drive aperture and disposed into one of the two, bone segments, and being operable, depending on the location of the fastener within the aperture, to selectively and independently to urge compression and distraction of the bone segments along the corresponding drive axis of the selected apertures,
whereby the universal surgical plate may be implanted on the segments of the bone regardless of which bilateral anatomical symmetry characterizes the bone.

2. The surgical plate of claim 1 further comprising a neck portion connecting the head portion and the body portion.

3. The surgical plate of claim 1 where the body portion of the plate further comprises:
an elongated drive aperture defined therein; and
at least two circular shaped anchor apertures defined therein.

4. The surgical plate of claim 1 where the plate includes a front and reverse side, and where the body portion of the plate further comprises a plurality of notches defined on the reverse side of the body portion that reduce surface contact between the plate and underlying bone.

5. The surgical plate of claim 1 where the head portion includes an end and further comprises a pair of recessed corners provided on the end thereof.

6. The surgical plate of claim 1 where at least two of the three elongated drive apertures defined in the head portion are arranged and configured to apply a compression force to the curvilinear cut in a direction 30 degrees with respect to either side of the longitudinal axis when the corresponding fastener is received therein.

7. The surgical plate of claim 3 where at least one of the three elongated drive apertures defined in the head portion and the elongated drive aperture defined in the body portion are arranged and configured to apply equal and opposing compression forces along the longitudinal axis when the corresponding fasteners are received therein.

8. The surgical plate of claim 1 where each drive aperture has a distal end and where the at least three elongated drive apertures defined in the head portion are defined within the head portion so that the distal end of each drive aperture is defined at a maximum distance from every other drive aperture distal end in the head portion.

9. The surgical plate of claim 3 where the at least three elongated drive apertures defined in the head portion and the elongated drive aperture defined in the body portion are each arranged and configured as means for providing compression on the curvilinear cut when coupled to the underlying bone when the corresponding fastener is received therein.

10. A method of implanting a universal surgical plate during a tibial plateau leveling osteotomy (TPLO) procedure in either the left or right rear leg of a canine comprising:
making a curvilinear cut in a left or right tibia to define at least two bone segments;
inserting a jig pin into one of the at least two bone segments of the tibia above the curvilinear cut for coupling to a surgical fixture to stabilize the corresponding bone segment;
coupling the universal surgical plate to the stabilized segment of the left or right tibia close to the jig pin and the other of the at least two bone segments with a plurality of orthopedic screws and straddling the curvilinear cut; and
applying a selectively directed compressive force 30 degrees with respect to a longitudinal axis of the surgical plate across the curvilinear cut by fastening an orthopedic screw into the stabilized segment into a selected one of two drive apertures which are symmetrically defined in the plate with respect to the longitudinal axis of the plate, which selected one of the symmetrically defined apertures is selected depending on whether the plate is implanted onto the left or right tibia.

11. The method of claim 10 where coupling the surgical plate to the tibia close to the jig pin comprises coupling a recessed corner of the surgical plate substantially close to the jig pin.

12. The method of claim 10 where coupling the surgical plate to the tibia comprises:
inserting at least two compressive drive orthopedic screws above the curvilinear cut;
inserting at least one anchor orthopedic screw above the curvilinear cut;
inserting at least one compressive drive orthopedic screw below the curvilinear cut; and
inserting at least two anchor orthopedic screws below the curvilinear cut.

13. The method of claim 10 where applying a directed compressive force 30 degrees with respect to a longitudinal axis of the surgical plate comprises applying a directed compressive force 30 degrees to the left of the longitudinal axis of the surgical plate when the surgical plate is implanted on the right rear leg of the canine as seen from a front side of the plate with a head of the plate at the top.

14. The method of claim 10 where applying a directed compressive force 30 degrees with respect to a longitudinal axis of the surgical plate comprises applying a directed compressive force 30 degrees to the right of the longitudinal axis of the surgical plate when the surgical plate is implanted on the left rear leg of the canine as seen from a front side of the plate with a head of the plate at the top.

15. The method of claim 10 further comprising applying a directed compressive force along the longitudinal axis and an equal and opposite directed compressive force along the longitudinal axis.

16. A method of implanting a universal surgical plate having a longitudinal axis for treating a bone fracture in a canine wherein the bone has bilateral anatomical symmetry comprising:
coupling a head portion of the universal surgical plate to the bone with a plurality of orthopedic screws on a side the fracture which is characterized by a left or right bilateral anatomical symmetry by selectively fastening to the bone through a selected one of at least two symmetrically defined angularly oriented drive apertures in the head portion, which drive apertures are symmetrically defined with respect to the longitudinal axis;
disposing a neck portion of the universal surgical plate across the fracture; and
coupling a body portion of the universal surgical plate to the bone on an opposing side of the fracture with a plurality of orthopedic screws.

17. The method of claim 16 where coupling the head portion of the surgical plate with a plurality of orthopedic screws comprises inserting at least one compressive force orthopedic drive screw into the surgical plate so that a directed compressive force 30 degrees from the longitudinal axis of the body portion is applied to the bone.

18. The method of claim 16 where coupling the head portion of the surgical plate with a plurality of orthopedic screws comprises inserting at least one compressive force orthopedic drive screw into the surgical plate so that a directed compressive force along the longitudinal axis of the body is applied.

19. The method of claim 16 where coupling the body portion of the surgical plate with a plurality of orthopedic screws comprises inserting at least one compressive force orthopedic drive screw into the surgical plate so that a directed compressive force along the longitudinal axis of the body is applied.

20. The method of claim 16 further comprising reducing the surface contact between the surgical plate and underlying by means of a plurality of notches defined into the reverse side of the body portion.
